(19) United States
(12) Patent Application Publication
(10) Pub. No.: US 2022/0262517 A1
(43) Pub. Date: Aug. 18, 2022

(54) SYSTEM AND METHOD FOR MONITORING ENERGY USAGE TO ANALYZE PATIENT HEALTH

(71) Applicant: Reciprocal Labs Corporation, Madison, WI (US)

(72) Inventors: DAVID VAN SICKLE, Madison, WI (US); ROBERT LOUIS BADDELEY, Madison, WI (US); NICHOLAS JOHN HIRONS, Madison, WI (US)

(21) Appl. No.: 17/630,729

(22) PCT Filed: Jul. 31, 2020
(86) PCT No.: PCT/US2020/044584

§ 371 Date: Jan. 27, 2022

Related U.S. Application Data
(60) Provisional application No. 62/881,024, filed on Jul. 31, 2019.

(51) Int. Cl. G16H 50/20; H02J 13/00

(52) U.S. Cl. CPC G16H 50/20; H02J 13/00001; H02J 2310/14; H02J 2310/70; H02J 13/00002

(57) ABSTRACT

A system and method for monitoring the health of a patient in a residential environment. An energy monitoring module is in communication with an electrical panel providing power to electrical devices in the residential environment. The energy monitoring module is operable to monitor energy consumed by the electrical devices. A data energy analysis engine collects energy data from the energy monitoring module over a period of time. A health data correlation engine determines a health condition of the patient based on the collected energy data.

### PRIORITY CLAIM

This application claims priority to and benefit of PCT Application No. PCT/US2020/044584, filed Jul. 31, 2020, which claims priority to and benefit of U.S. Provisional Patent Application Ser. No. 62/881,024, filed on Jul. 31, 2019, which are both hereby incorporated by reference herein in their entirety.

### TECHNICAL FIELD

The present disclosure relates generally to health monitoring systems, and more specifically for collecting energy usage data from a residential environment to monitor patient health.

### BACKGROUND

Many people have recurring ailments that do not require hospitalization. However, such conditions may worsen between visits to health care professionals. For example, respiratory ailments such as asthma or chronic obstructive pulmonary disease (COPD) often may become more severe over a period of time. Asthma may cause wheezing, chest tightening, shortness of breath, and coughing. Asthma may be caused by an oversensitivity to inhaled substances that causes the bronchial airways to constrict and tighten. Such conditions could be either prevented or treated in a timely manner if a patient's health could be monitored on a continuous basis. Certain patients may be assigned home health care providers to perform checks on a daily basis. However, the expenditures for such personnel is often prohibitive. Further, in many cases, there is no need for such expenses. A series of health care monitors may also be carried by a patient at home. However, such monitoring devices are cumbersome and often a patient must be responsible for correctly operating such devices.

Thus, since there is no convenient method to continually monitor patient health in settings other than hospitals or care facilities, often preventive techniques or treatments cannot be applied in a timely manner. Further, a patient may not realize the onset of a worsening condition, and as a result, fails to consult a health care professional. Often, by the time a health condition has become noticeably severe to cause the patient to seek medical attention, more expensive and time consuming treatments must be applied.

There is a need for a system that allows for continuous monitoring of health conditions in a home environment without additional health related sensors. There is also a need for a system that uses energy data from a home correlated with health. There is also a need for a system that learns baseline energy usage data and correlation of the energy usage data with the health condition of a patient.

### SUMMARY

The present disclosure relates to a health monitoring system that analyzes energy usage data from electrical devices in a residential environment. The system uses an energy monitoring device to collect energy data. The energy data is analyzed to determine energy related signatures for different devices in the residential environment. Such information may be analyzed to track an individual patient's use of devices in the home, such as appliances, lights, care devices, to characterize “normal” usage and correlate the usage with the health condition of the patient. The system allows the detection of any “abnormal” usage that might be indicative of emerging health risks. The system does not require any specialized monitors that need to be attached to the patient in the home environment.

One disclosed example is a system for monitoring the health of a patient in a residential environment. An energy monitoring module is in communication with an electrical panel providing power to a plurality of electrical devices in the residential environment. The energy monitoring module is operable to monitor energy consumed by the plurality of electrical devices. A data energy analysis engine collects energy data from the energy monitoring module over a period of time. A health data correlation engine determines a health condition of the patient based on the collected energy data.

A further implementation of the example system is where the health condition includes a change in sleep quality or duration of the patient. Another implementation is where the health condition includes a change in diet or appetite of the patient. Another implementation is where the health condition includes a change in physical activity or mobility of the patient. Another implementation is where the plurality of devices includes a therapeutic device for treatment of the patient. Another implementation is where the health condition is a respiratory condition. Another implementation is where the energy data includes a pattern based on electrical usage. Another implementation is where the energy data includes the times when each of the plurality of devices is turned on. Another implementation is where the energy data includes determination of a specific device signature for each of the plurality of devices. Another implementation is where the specific device signature is determined from machine learning. Another implementation is where the specific device signature is determined from a rule set of device signatures. Another implementation is where the correlation between the health condition and the specific device signature is determined from machine learning. Another implementation is where the energy data includes the total electrical usage of the plurality of devices over a period of time.

Another example is a method for monitoring health of a patient in a residential environment. Energy use data of at least one device in the residential environment is collected. An energy use pattern is determined from the energy use data. The energy use pattern is correlated with a health condition of the patient.

A further implementation of the example method is where the health condition includes a change in sleep quality or duration of the patient. Another implementation is where the health condition includes a change in diet or appetite of the patient. Another implementation is where the health condition includes a change in physical activity or mobility of the patient. Another implementation is where the device is a therapeutic device for treatment of the patient. Another implementation is where the health condition is a respiratory condition. Another implementation is where the energy use pattern is a pattern based on electrical usage. Another implementation is where the energy use pattern includes the times when each of the plurality of devices is turned on. Another implementation is where the energy data includes determination of a specific device signature for each of the plurality of devices. Another implementation is where the method includes determining the specific device signature from machine learning. Another implementation is where the method includes determining the specific device signature from a rule set of device signatures. Another implementation is where the method includes determining the correlation between the health condition and specific device signature from machine learning. Another implementation is where the energy use pattern includes the total electrical usage of the device over a period of time.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 is a block diagram of a home energy monitoring system to provide energy use data for determining patient health;

FIG. 2 is a block diagram of a health analysis system that analyzes energy data collected by the monitoring system in FIG. 1;

FIG. 3A is a graph showing example weekly energy output from the home monitoring system in FIG. 1 that may be used by the health analysis system in FIG. 2;

FIG. 3B is a graph showing example daily energy output from the home monitoring system in FIG. 1 that may be used by the health analysis system in FIG. 2;

FIG. 4 is an example interface showing the use times of different devices based on the data collected by the monitoring system in FIG. 1;

FIG. 5A is an example of pattern collection for learning recognition of energy use from different devices;

FIG. 5B is an example of the learning routine using base line pattern to identify devices based on energy data;

FIG. 6 is a flow diagram of a learning routine that learns energy usage patterns in relation to electronic devices and creates models of the use for analysis of live sensor data;

FIG. 7A is a table of training data for training the learning routine in FIG. 6;

FIG. 7B is a flow diagram of the training process for a learning routine using the training data from the table in FIG. 7A;

FIG. 7C is an example diagram of a neural net to determine energy patterns for a device;

FIG. 8 is a flow diagram of a learning routine that learns energy usage patterns in relation to health conditions of an individual patient and creates models of the use for analysis of live sensor data; and

FIG. 9 is a block diagram of a health care system that may use the health monitoring data from the system in FIG. 2.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a health monitoring system that is based on analysis of energy usage data from a home environment of a patient. The health monitoring system uses an energy monitoring device coupled to the main power of a home to collect energy usage data. The energy monitoring device may be a smart energy meter, or another type of electricity monitoring device, either locally or centrally installed in the home setting. The energy monitor is used to track, process, store and communicate the electricity history of the residential environment. The data is analyzed to determine the electricity signatures of specific devices in the home. Such information may be analyzed to track an individual patient's use of such devices, including appliances, lights, and treatment devices such as respiratory care devices. The usage by an individual patient is used to characterize “normal” usage correlated with the health condition of the patient. The continual collection of energy data may be used to detect any “abnormal” usage that might be indicative of emerging health risks for the patient.

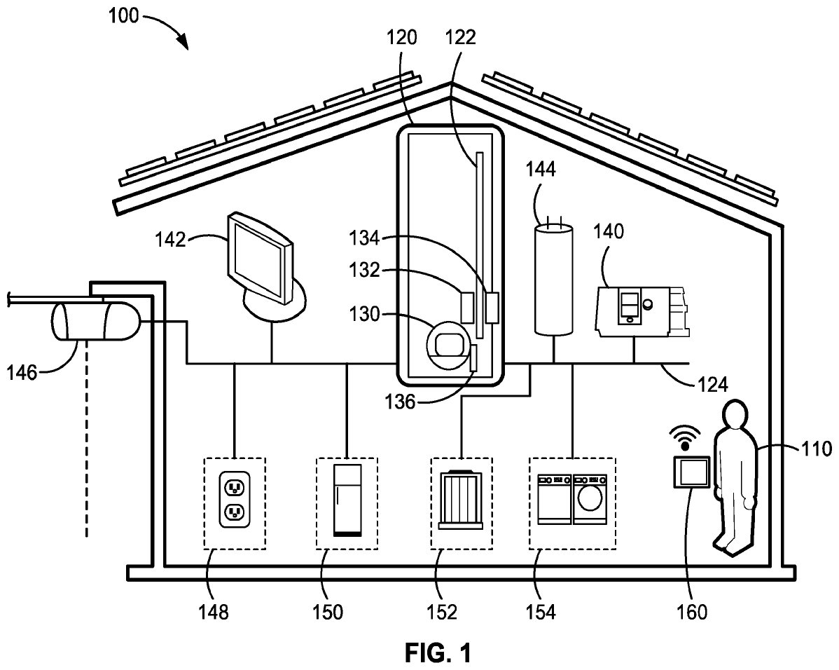

FIG. 1 shows a residential environment such as a home 100 for a patient 110. The residential environment may be a house, an apartment, or other building associated with the patient 110. The home 100 is connected to an external power source, such as an electrical utility company that provides a power line that is connected to the home 100 through an electrical junction panel 120. The electrical junction panel includes main power leads 122 that feed power to the home 100. The junction 120 supplies electrical power from the main power through a wiring network 124 to different rooms in the home 100. The junction 120 has an attached energy sensor module 130 that collects energy usage data.

In this example, the energy sensor module 130 is a Sense Monitor® manufactured by Sense Labs Inc. of Cambridge Mass. The energy sensor module 130 collects and sends energy data to an external device such as a smartphone or computer. The sensor module 130 includes two sensors 132 and 134, that may be current transformers, which clamp around the main power leads 122 feeding the home 100. The sensor module 130 also includes an antenna 136 for wireless transmission of the energy data. In this example, the sensor module 130 is centralized and monitors power from every electrical device in the home 100.

In this example the sensor module 130 may include a wireless link to an external client device 160 such as smart phone or tablet. The wireless link may incorporate any suitable wireless connection technology known in the art, including but not limited to Wi-Fi (IEEE 802.11), Bluetooth, other radio frequencies, Infra-Red (IR), GSM, CDMA, GPRS, 3G, 4G, W-CDMA, EDGE or DCDMA200 and similar technologies. It is to be understood that a series of decentralized sensors such as sensors on each electrical device in the home (either integrated in the device or a modular unit) may be used in conjunction with the external device that may collect all data including that from the sensor module 130 and consolidates the data. In addition, different energy sensors may provide additional data to the sensor module 130 regarding energy use. For example, certain appliances in the home 100 may use protocols such as the Internet of Things (IOT) to communicate energy data. Other devices such as a NEST device or a digital assistant in the home 100 may collect discrete energy data for certain devices and send the data to the sensor module 130 or an external device.

The junction panel 120 supplies power to different appliances and devices 140, 142, 144, 146, 148, 150, 152, and 154. The devices 140, 142, 144, 146, 148, 150, 152, and 154 may represent either individual devices or groups of devices in the home 100. The devices and appliances may include a home care treatment device such as a CPAP device or respiratory therapy device represented by device 140. The appliances may also include entertainment devices 142, a water heater 144, a garage door opener 146, lights and outlets 148, food preparation and storage devices 150, a heating ventilation and air conditioning (HVAC) unit 152, and washers and dryers 154.

The energy sensor module 130 monitors the power from the main power line 122 feeding into the electrical junction 120. The energy sensor module 130 collects millions of readings every second from the changes in electrical current and voltage. Based on this high-resolution data, advanced machine learning algorithms may be used to identify what devices 140-154 are drawing power from unique changes in electrical current and voltage during operation. For example, a conventional light bulb may have a signature that draws a lot of current as the filament heats up and then stabilizes. The current and voltage are in phase with each other and thus the signature may be characterized as a resistive load. In contrast, a microwave signature may include an initial surge as the microwave charges up and a second surge when the magnetron is activated.

In this example, the collected energy data and resulting specific device energy signatures may be displayed on a user interface generated by an external computing device 160 that may be operated by the patient 110. The remote external device 160 may be a portable computing device such as a smart phone or tablet that executes an application to collect and analyze data from the monitor 130. The application for energy monitoring on the remote external device 160 may display the times each device is running and other information such as energy use.

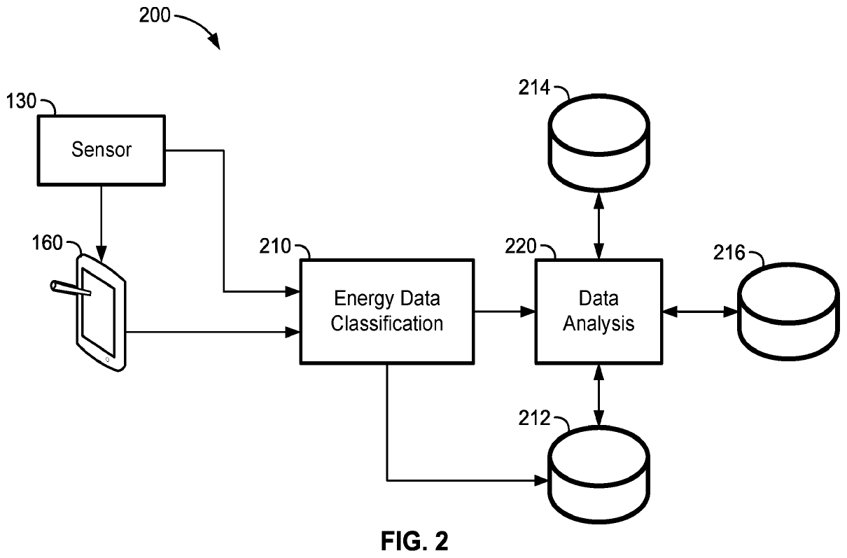

FIG. 2 is a block diagram of a health monitoring system 200 that may incorporate the energy data collected by the energy sensor module 130 and analyzed by the external computing device 160 for health monitoring of the patient 110. Alternatively, the system 200 may collect data directly from the energy sensor module 130 via a wireless link and perform the energy analysis functions described above.

The health monitoring system 200 determines the use, timing and sequence of the power use of residential elements such as lighting, entry/exits, heating/cooling, appliances (refrigerator, washing machine, dishwasher, etc.), and other supporting household objects, including medication delivery devices and durable medical equipment. The use, time and sequence of such devices may be correlated to a daily or weekly pattern of device use and activity patterns for the patient 110. In addition to active, goal-oriented electricity use, background, always-on current can be measured to help infer the characteristics/profile of the residence, such as type of dwelling, socioeconomic status, and presence of features such as internet connectivity. Other examples may include analysis of characteristics of residential environments. For example, homes with central heating and air conditioning will have different characteristics from homes without central heating and air conditioning. Allergy season may impact a patient without air conditioning more than a patient with air conditioning. Thus, various contextual aspects could be important inputs into estimates of risk or potentially could assist in shaping or targeting specific interventions. For example, the housing type may have an impact on asthma incidence and morbidity due to its effects on indoor air pollution exposures.

The system 200 collects the energy data from the energy sensor module 130 and classifies the energy data via an energy data classification module 210. The classified energy data is stored in an energy database 212. The system 200 ascertains and establishes a series of index patterns of use and characterizes and associates those in relation to the current disease severity, acuity and activity, and management of the patient 110 via a patient analysis engine 220. The analysis engine 220 is coupled to the energy database 212 and a patient health database 214. The patient health database 214 stores current health conditions and other demographic information of the patient 110 may be determined by other means (e.g. surveys, clinical examinations, biomarkers and physiological measurements, pharmacy records, and other information). Such information may be collected and stored in various individual databases that may be accessed by the system 100. The analysis engine 220 determines the relation between patient health and energy use data taking into account the correlation between patterns of energy use and the health condition of the patient 110.

One example of monitoring a health conditioning may be measuring the status of chronic (respiratory) disease of a patient. The information indicating a change in health condition related to such a disease may be used to provide prospective information about the burden and management of the respiratory disease to the patient. The database 214 may include information about the patient that has a respiratory disease. The analysis engine 220 in FIG. 2 may provide monitoring on respiration based on use of electrical devices on different floors of the building, which may indicate weakening condition (less use of devices on a second floor) or a strengthening condition, evidenced by greater activity or movement. The analysis engine 220 may also monitor use of therapeutic devices such as a CPAP device, an oxygenator, or a ventilator. The analysis engine 220 may also infer greater or less physical activity based on frequency and speed of movement within the building, or use of specific equipment, such as a treadmill. The condition of the patient may also be correlated to other diseases or potential diseases such as fall risk or dementia

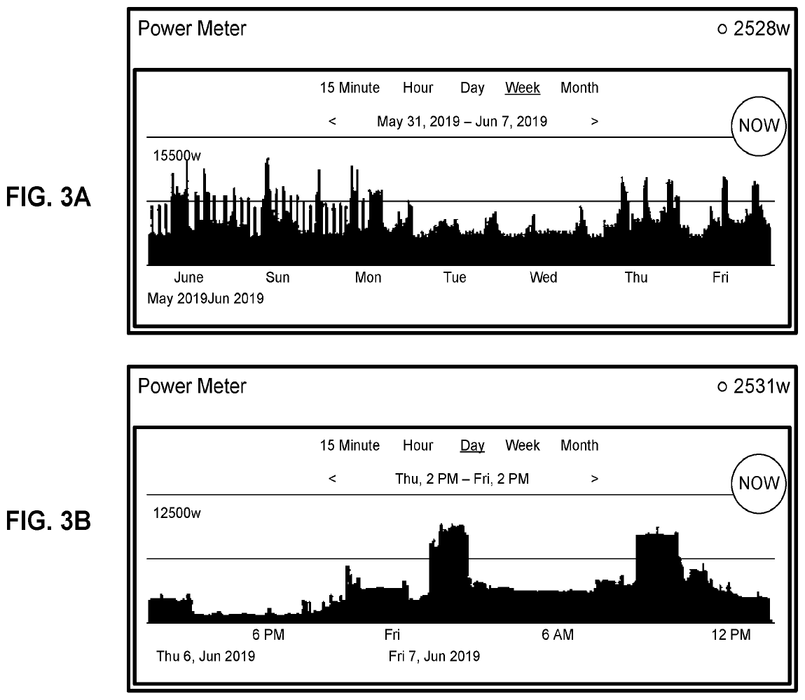

The analysis engine 220 may use different approaches to correlate energy usage with health status of a patient. One such approach analyzes electricity use over a period of time, for example, a weekly or daily history of use. The electrical data output from the energy monitoring module 130 in FIG. 1 may thus be analyzed on a periodic basis, such as either on a weekly or daily basis. FIG. 3A is an example graph of electrical use data for a home collected by an energy monitor module over the course of a week. FIG. 3B is an example graph of electric use data for a home collected by an energy monitor module over the course of a day. The data collected during a certain period may be analyzed mathematically in a multitude of ways to determine the relationship of parameters such as total watts, number of peaks, number of peaks per day, timing of peaks, amplitude, and so on, to health conditions such as disease status and impairment for an individual patient. For example, significantly reduced total watts could indicate an emergency in which the patient has not gotten out of bed. The timing of peaks and their number may indicate patterns of behavior such as food preparation or other daily rituals. Such data is analyzed by the energy data classification module 210 and the results are stored in the database 212 for the analysis engine 220.

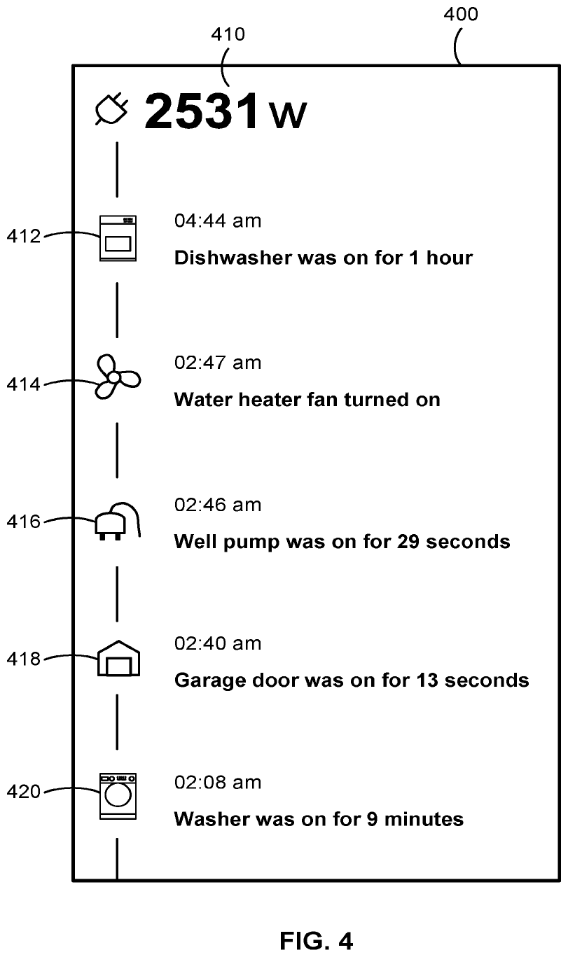

A second approach monitors the individual activity and timing/sequence of use of appliances such as dishwashers, washer/dryers, microwave ovens, television sets, and coffee makers, among many other devices in the home 100 shown in FIG. 1. The timing when such devices are used may be logged and displayed as shown in FIG. 4. FIG. 4 is an example graphical interface 400 that shows different appliances and the length of time they are turned on. In the example interface 400, the total power 410 is displayed for a certain period of time. A series of icons 412, 414, 416, 418 and 420 represent different devices and the times such devices are turned on. The data used to generate the interface 400 may be determined by the energy analysis engine 210 and used by the analysis engine 220 to monitor the timing of the devices and correlate the data with the health of an individual patient 110. This method may also associate these appliances with specific rooms or regions of the home 100, to create general and specific topography of indoor movement of the patient 110 over one or more periods. As explained above, for respiratory ailments, this may be indicative of improvement of a patient resulting in more activity or an increase in the severity of the ailment indicated by less use and movement.

Other methods for energy usage analysis may be employed by the analysis engine 220 to correlate with health conditions. For example, the total energy load during the day, across one or more devices, or a particular collection that is matched to a disease profile. In addition, timing of load, across one or more devices or a particularly informative collection of devices, that matters to disease status. Another factor may be frequency of activity, or timing of various devices (and considering timing windows) over the course of a day. A sudden drop in power to zero, as indicated by a power outage, may trigger the analysis engine 220 to request assistance on behalf of the patient if they are on any kind of medication or treatment that requires constant electrical connection.

The analysis engine 220 may also determine probability of use, or probability of a pattern for a patient, given additional data inputs. For example, knowing that the patient has had an exacerbation or change in their disease status, the analysis engine 220 may predict ways in which energy use of the patient will change. For example, it is less likely a patient with an exacerbation of a disease will be mobile or do their laundry. Thus, it can be expected that certain patterns, such as using the bathroom, to take longer or be more frequent. With this disease data specific to the patient, a feedback loop may be avoided in which the analysis engine 220 detects changes in the energy use patterns and indicates a problem to a caregiver that they already know about.

Further, knowing that there is a deviation in the pattern, the analysis engine 220 can determine the probability that the disease state has changed, how, and what impacts that will likely have on the patient as well as their continued energy use. For example, noticing that the patient is using their medical devices more or longer could indicate an exacerbation is about to happen, so the analysis engine 220 is primed to detect one more easily. The analysis engine 220 may explain the probability of disease status=X, updated based on emerging events in the electricity use profile as it unfolds during the day. Such predictions may be enhanced by additional data from other patients with similar demographics as the patient. The analysis engine 220 may also integrate information from cooperating data services, such as voice interfaces, digital home assistants, smart plugs and connected appliances, calendars, computer networks, and mobile phones that may be associated with the patient 110 or be present in the home 100. Such data may be accessed through third party databases 216.

A third approach is based on routines of daily life that are determined through composite electricity and object use signatures associated with important functional routines of daily life, especially those with an established or plausible relationship to disease or health status. For example, sleep quality and timing may be determined from the time lights are turned on and off, when a water heater is activated, and when kitchen appliances are used. This data allows the analysis engine 220 to directly or indirectly detect or determine awakening and sleeping, the diurnal timing of sleep, the length of time asleep, the degree of sleep disturbance, and other parameters of sleep quality and duration for the patient. Another routine that may be determined by device energy signatures may be a meal routine for a patient. The system may determine the relationship between the presence or absence of a meal routine, which might indicate or proxy for appetite, mental status, or functional ability. For example, in the case of a breakfast routine detecting activation of food preparation appliance 150 in FIG. 1, such as a coffee grinder, coffee maker/kettle, microwave, toaster, and to assess correspondence with disease status.

Other Activities of Daily Living (ADLs) may be determined. For example, macro-mobility events may be determined such as being in and out of the home via garage door activity or activating or deactivating a home alarm. Micro-mobility activity may be determined based on factors such as number of rooms with intentional electric/appliance activity, and variability/regularity compared to a base index for the patient. Another activity, may be determining bathing through hot water heater activity, and activating lights in bathroom, etc. Other activities may be determined such as monitoring a dishwasher, and washer dryer, either directly via appliance load or indirectly via load from hot water heater.

This data may be used as an indicator of fitness for unassisted living for a particular patient. The data may be used as an indicator of specific tasks a patient is struggling with so that assisted care can be focused. The data may indicate incontinence or obstruction. The data may indicate general cleanliness, which could lead to other issues like infection or food-related illnesses. The data may show signs of dementia (e.g., washing clothes too frequently, or running the dishwasher repeatedly, or leaving the house when at unexpected times. The data may be used as an indicator of an emergency. For example, if the energy usage of devices is significantly lower than normal, the patient may be unconscious or struggling. The data may be used to determine the patient is not in the residential environment (e.g., via data from the garage door or home alarm) and thus allow the analysis engine 220 to discount deviations in daily patterns.

The system also allows monitoring of home medical equipment and monitoring of treatment using such devices such as the device 140 in FIG. 1. For example, such devices for respiratory therapy may include portable oxygen concentrators, non-invasive ventilators, CPAP machines, and nebulizers. When installed, the energy monitor module 130 in FIG. 1 allows the remote and passive collection of data about the use of devices for different treatments. For example, the energy signatures of devices such as portable oxygen concentrators, non-invasive ventilators, CPAP machines, and nebulizers may be monitored for respiratory care and treatment.

In such an example for respiratory care and treatment, the voltage/load signature of therapeutic medical equipment, such as nebulizers, or oxygen concentrators, is monitored. When a device, such as a nebulizer, is powered on and used to deliver aerosolized medications, the timing of this event and its duration is captured and stored from the energy monitor module 130. This allows an assessment of treatment adherence by measuring actual use against a prescribed regimen. Furthermore, by calculating the duration of use some estimate of administered medication dose may be estimated. In the case of nebulized emergency medications, such as albuterol, and other devices such as oxygen or non-invasive ventilation, measures of the frequency and time of day of use would provide an estimate of risk and impairment.

The analysis engine 220 may also examine the voltage signature resulting from battery charging, and monitors the periodic recharging of important medical devices that have portable embodiments, such as nebulizers and portable concentrators, and durable equipment, such as wheelchairs. Such voltage signatures are indicative of the use of such devices.

The system 200 collects electrical data and outputs personal baselines for energy use on different devices or overall usage. The baselines may be used to compare current data to monitor for permutation(s) from the personal baselines described above that would suggest a change in disease status, treatment patterns, or quality of life. These signals of emerging impairment or risk could include signals that indicate changes in physical activity and mobility, sleep quality or duration, diet and appetite, and other physiological significant factors. Deviation from their normal routines, or timing of such routines, as established by the above activities monitored in daily life.

Such changes could suggest emerging health issues or diminished ability to participate in activities of daily living, which result from increasing dyspnea or an impending exacerbation. Other domestic indicators such as the opening or closing of garage doors could indicate changed mobility patterns or increased social isolation. The data from the system may be used by health care providers to alter treatment or anticipate changes and recommend predictive care. Further, the data from the system may be used to alert authorized users and other systems to the existence and nature of the deviation, severity/certainty of disturbance.

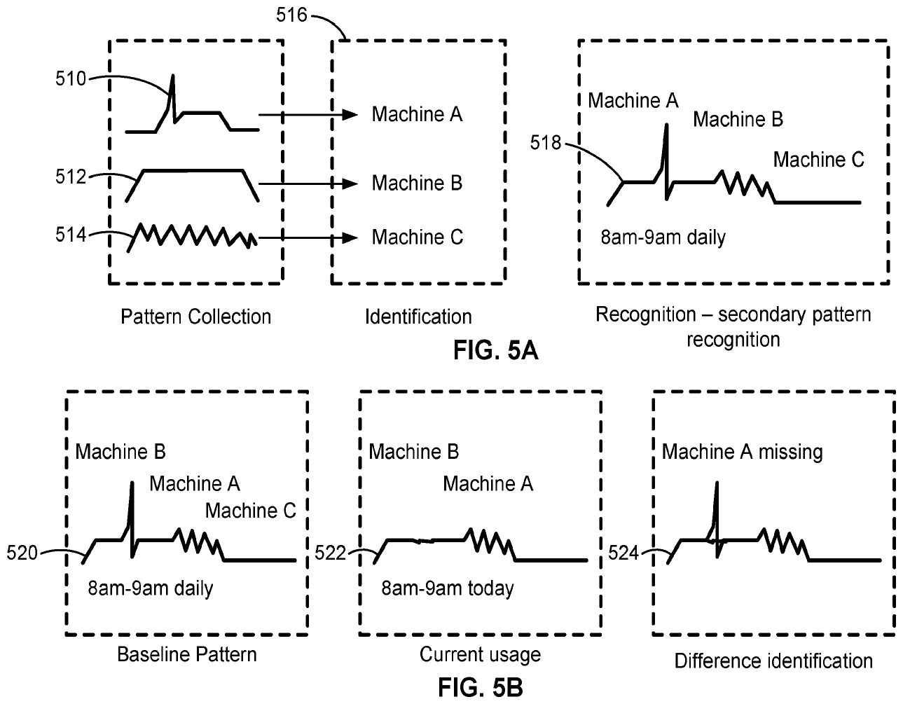

FIG. 5A is an example of patterns for the process of learning pattern recognition from energy use from different devices. FIG. 5A shows a collection of energy patterns 510, 512, and 514 from energy use of different devices. The energy patterns may be obtained from energy data from individual devices. The patterns 510, 512, and 514 may be identified with different types of devices A, B, and C (516). In this example, the individual patterns 510, 512, and 514 may be collected by an energy monitor as deviations from single line. One device may be turned on at a time to collect the pattern of the device and then the device is turned off. One example of the process of pattern collection and identification is an interactive interface where the monitor such as the energy sensor module 130 in FIG. 1 queries a user through the remote external device 160 each time a new pattern is detected. The application may access a database of known patterns to suggest identification of a device. The application may rely on the user to input the identification of the specific device to associate with that pattern. An overall energy consumption trace 518 may be used in conjunction with the energy patterns 510, 512, and 514 to identify when a particular device is used. This process may be used for secondary pattern recognition.

FIG. 5B is an example of using the patterns learned in the process described above for different identification of devices from overall energy use. A base line pattern 520 of energy use is provided, that shows the overall energy use from different devices. The base line pattern 520 is compared to a current usage pattern 522 over the similar period of time. The comparison of the base line pattern 520 and the current usage pattern 522, may be used for different identification 524. In this example, the difference between the base line pattern 520 and 522 allows a determination that one device (device A) was not used during the current usage period.

The energy usage patterns, such as patterns 510, 512, and 514, may be determined by machine learning. This process limits any need for a patient to generate their own energy data by turning devices on and off sequentially at the start of the installation. The machine learning process may also be continually refined by real-time data obtained from monitors such as the energy sensing module 130 in FIG. 1. Thus, the model generated by the machine learning process gets better over time. Synthetic data generation (e.g., randomly adding device signals for different periods and adding random noise that is similar to real historical data to simulate an infinite number of residential environments) and new device detection allow for continuous improvement only possible with deep learning models. An alternative to the machine learning generated models may be a fixed rule set for identifying energy patterns for specific devices.

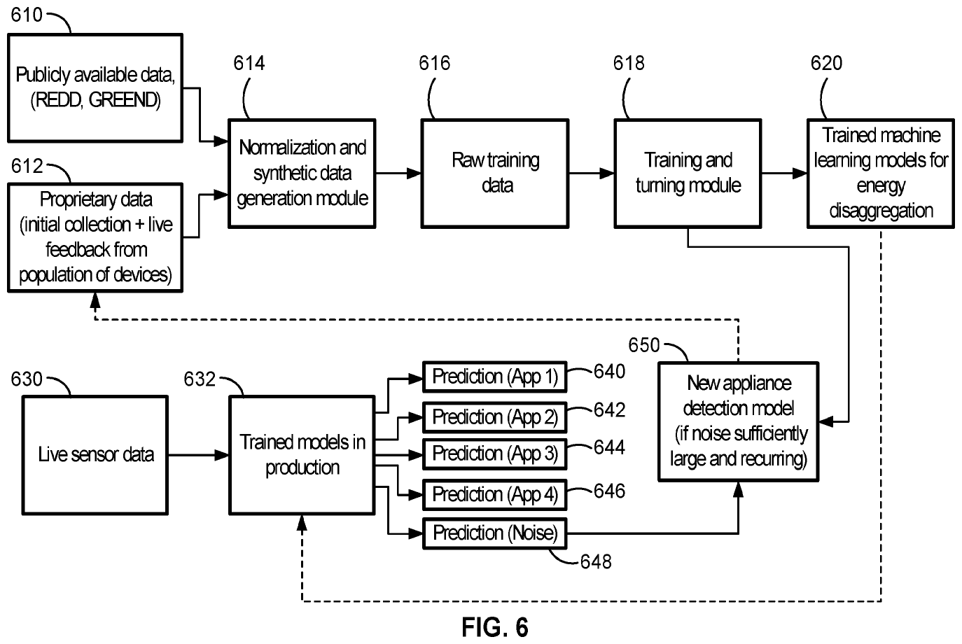

FIG. 6 is a flow diagram of a routine that learns energy usage patterns in relation to electronic devices such as those in FIG. 1 and applies the learned model to analyze live energy data from an energy monitor. The learning routine relies on publicly available data 610. Such data may include publicly available household energy consumption datasets such as the Reference Energy Disaggregation Data Set (REDD) and the GREEND electrical energy data set. Additional data may be obtained from proprietary sources 612 such as collection and live feedback from a population of devices monitored by energy sensing modules such as the energy sensing module 130 in FIG. 1 from multiple patients. A normalization and synthetic data generation module 614 receives the public and proprietary data 610 and 612. The module 614 first normalizes the data, for example unifying appliance labels, and upsampling or downsampling time steps to the desired frequency that will be used in production. It may then generate synthetic data by creating synthetic households from randomly selected devices over randomly selected periods of time, and creating a household level load time series by adding all synthetic device loads and some representative level of noise. Raw training data 616 is produced from the module 614. A training and tuning module 618 is provided with the raw training data 616. The training and tuning module 618 produces a set of trained machine learning models for energy disaggregation 620.

Once the models are sufficiently accurate, the models may be used by the analysis engine 220 in FIG. 2. A set of live sensor data 630 is collected continuously from monitors such as the energy sensing module 130 in FIG. 1. The live sensor data is input to the trained models (632) that are used by the energy analysis engine 210. The energy analysis engine 210 employs the trained models to predict or classify the use of different devices based on the received sensor data. Each example known device has a separate prediction module that outputs predictions of energy use 640, 642, 644, and 646, based on the received sensor data. The output predictions of energy usage are then output as the usage for the devices in the particular residential environment associated with the sensor data. Additional unidentified sensor data may be classified as noise (648). This noise data may be collected to create a new device detection model 650. Such a model may be created if the noise data is sufficiently large and recurring. The model is created by the training and tuning module 618. The new device model may be added to the proprietary data 612 to updated the known models of devices.

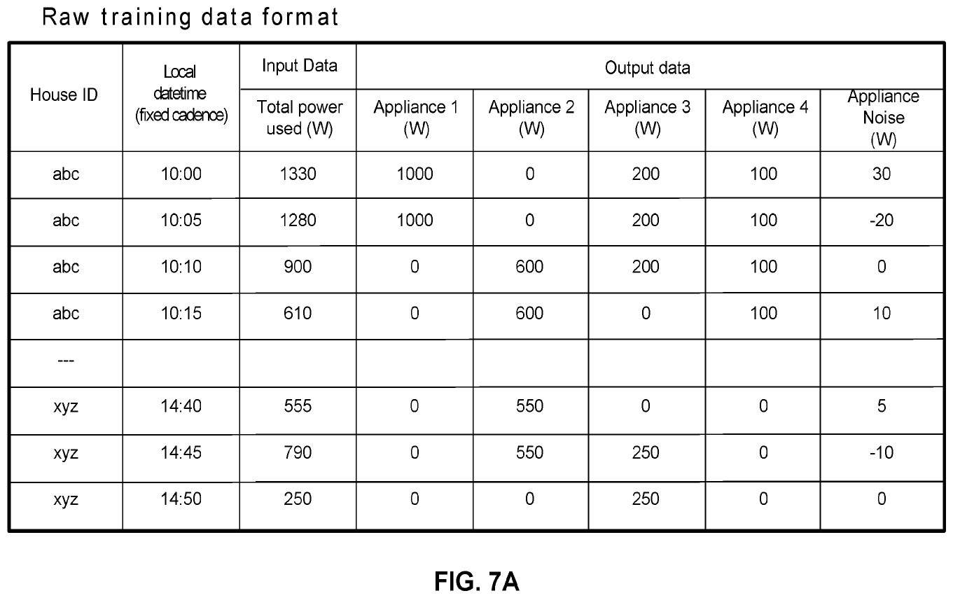

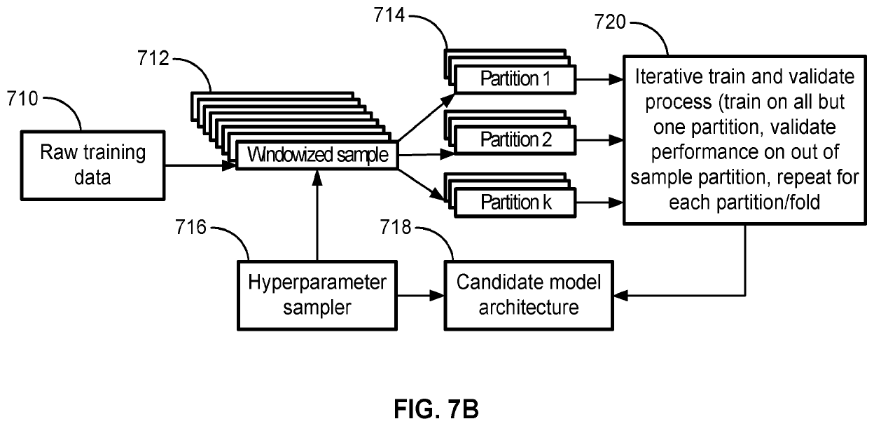

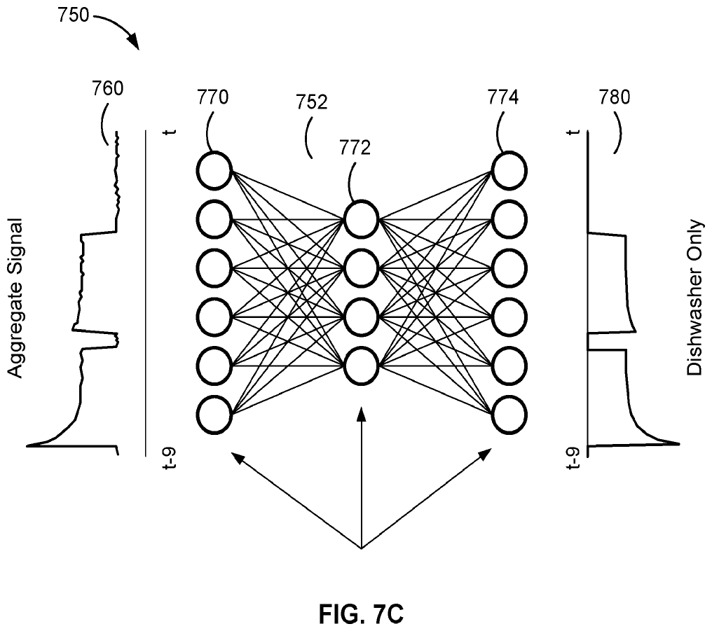

FIG. 7A is a table of training data for the routine in FIG. 6. As may be seen in FIG. 7A, the training data includes a house ID, and a local date and time associated with each change in energy. The input data is the total power use for each time. The output data is the use and corresponding time of each device in the set as well as random noise data. As explained above, the training data is used to create the model and teach the proper weighting for the training and tuning module 618 to identify different devices by their energy patterns.

FIG. 7B is a flow diagram of the training process for a learning routine using the training data from the table in FIG. 7A. The process is employed by the training and tuning module 618 to refine the models 620 in FIG. 6. A set of raw training data 710 such as the data in the table shown in FIG. 7A is collected. The training data is used to create a set of windowized samples 712, such that each sample contains multiple time steps of household-level loads as input and either the current time step or multiple time steps of a device level load as an output. For example, if there was a desired time window of 5 minutes and each time step was 30 seconds and the goal was to predict current time step for refrigerator use, the input would include 10 time steps of the household-level load and the output would include only the current time step of the refrigerator load. The size of a particular training window is a hyperparameter that may be tuned to optimize performance, and is typically related to the duration of a device signature. For example, the optimum window for a kettle may be 2 minutes, while the optimal window for a washer-dryer may be 30 minutes. The windowized samples 712 are used to create a series of partitions 714 that relate to different residential environments. These may include synthetic residential environments that will be kept out of sample. The partitions allow models to predict across many different kinds of residential environments and device combinations, so a portion of residential environments may be kept “out of sample” to measure performance and pick the best model structure. The windowized samples 712 are partitioned into k distinct groups of residential environments (partitions) based on random samples of different residential environments without the same residential environment appearing in different partitions.

A hyperparameter sampler 716 is used to both determine the windows for the windowized samples 712 as well supply parameters for a candidate model architecture 718. The candidate model architecture 718 is input into the neural network and used to assign initial weights. The training process for the neural network also uses the partitions 716 input to the model architecture 718 to iteratively train and validate the process (720). In this example, the process will train on all but one of the partitions 714. The process will validate the performance on the out of sample partition. This is repeated for each partition of the partitions 714. The results from the initial architecture are checked against the outputs from the training data to determine the accuracy of the predictions. If the predictions are sufficiently accurate, the model and corresponding weights are sent to production. If the predictions are not sufficiently accurate, the weights are adjusted and the process repeats with the adjusted model architecture.

FIG. 7C is a simplified diagram of an example neural net process 750 to output the energy patterns for one type of device such as a dishwasher. A neural net 752 is trained using the process described in FIG. 7B. An aggregate energy signal 760 is input into a series of initial nodes 770, each with a corresponding weight value. The initial nodes 770 are connected to a hidden layer of nodes 772 with different weight values. The output of the hidden layer of nodes 772 is output to a layer of output nodes 774. The output of the output nodes 774 filter an output representing an energy use pattern of the single device 780 such as the dishwasher in this example. The weights for the nodes of the neural net 752 are updated after each batch of samples to minimize a chosen metric such as mean squared error.

After the routine in FIG. 6 produces energy usage data for different devices, a routine may be run by the analysis engine 220 to monitor health conditions of the patient based on the energy usage data and other inputs. For example, for patients that have treatment devices that are monitored (inhaler, CPAP, oxygenator, ventilator), a training dataset may be created that links health indicators based on predicted appliance loads to health data. The data may be aggregated to a daily, weekly or monthly level. An indicator of an outlier or abnormality in individual device trends or a combination of device trends may be provided. The engine may determine period on period changes, for example, the difference or ratio of the past week versus the preceding week or comparable week from a prior period. The timing of powering certain devices may also be predictive of health conditions.

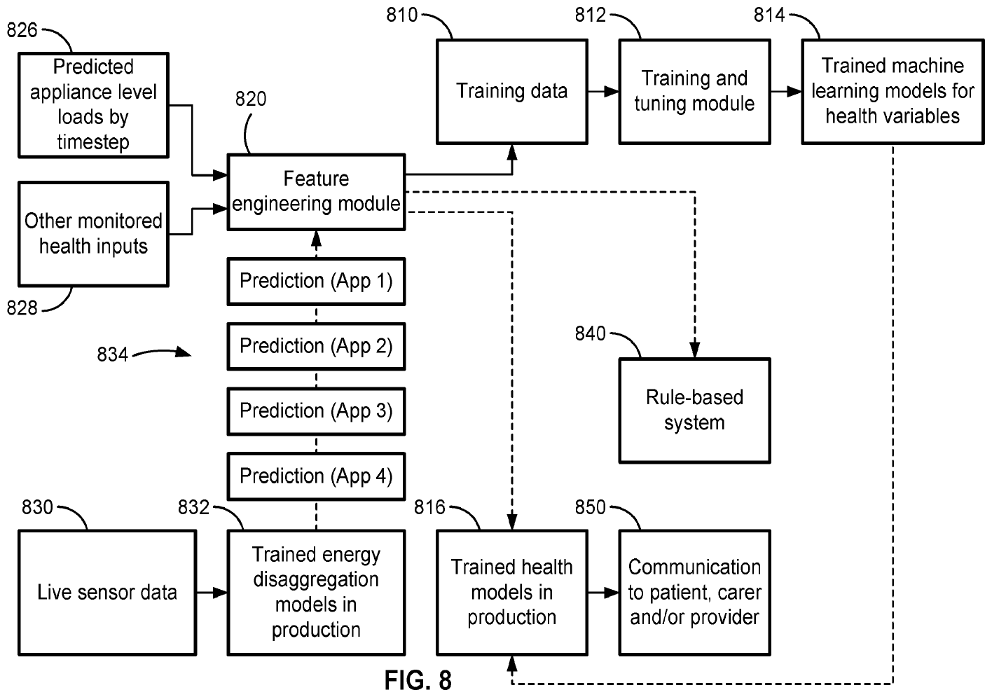

FIG. 8 is a flow diagram of a learning routine that learns energy usage patterns in relation to health conditions of an individual patient and applies the learned model to analyze live sensor data. The machine learning process is also continually refined by real-time data obtained from monitors such as the energy sensing module 130 in FIG. 1 and big data from other patients. Thus, the model for correlating energy usage to health conditions generated by the machine learning process gets better over time. An alternative to the machine learning generated models may be a fixed rule set for identifying health conditions based on energy patterns for specific devices.

A set of training data 810 is input to a training and tuning module 812. The set of training data includes a correlation with energy usage patterns of devices with health conditions for a patient. The training and tuning model 812 creates and refines trained machine learning modules for health variables 814. The trained modules 814 are refined for accuracy and when sufficiently accurate are moved to production models for predicting health 816. The process of training is similar to that explained above with reference to FIG. 7B.

An example feature engineering module 820 generates new input features from the predicted device load time series that are most relevant to determining health characteristics of the occupant for a particular patient based on several inputs. For example, the decline in current day lighting use compared to the same day a week prior may be predictive of an acute health issue resulting in an emergency doctor visit, while a month on month increase in TV usage during the middle of the day may be predictive of a slower but more chronic decline in activity and respiratory function. A set of live sensor data for overall energy use 830 is collected continuously from monitors such as the energy sensing module 130 in FIG. 1. The live sensor data is input to the trained models 832 determined by the process in FIG. 6 such as the predictions 640, 642, 644, and 646, that are used by the analysis engine 220. The analysis engine 220 employs the trained models to predict the use of different devices based on the received sensor data. Each example known device has a separate prediction module that outputs predictions of energy use 834 based on the received sensor data. The output predictions of energy usage are then output as the usage for the devices in the particular residential environment associated with the sensor data and provided to the feature engineering module 820. Data related to predicted device level loads by timestamp 836 is also provided to the feature engineering module 820. The data is obtained from the energy sensor modules such as the energy sensor module 130 in FIG. 1 after analyzed by the process in FIG. 6. Other monitored health input data 838 are also provided to the feature engineering module 820. For example, relevant data may be obtained from devices such as CPAP devices, inhalers, and portable oxygen concentrators. The output data from the feature energy module 820 is input into either the trained health models 816 or a rule based system 840 to output health conditions of the patient. The resulting health conditions may be communicated to the patient, care provider, or health care professional (850).

The flow diagrams in FIGS. 6 and 8 are representative of example machine readable instructions for collecting and analyzing energy data in FIG. 1. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithm is described with reference to the flowcharts illustrated in FIGS. 6 and 8, persons of ordinary skill in the art will readily appreciate that many other methods of implementing the example machine readable instructions may alternatively be used. For example, the order of execution of the blocks may be changed, and/or some of the blocks described may be changed, eliminated, or combined.

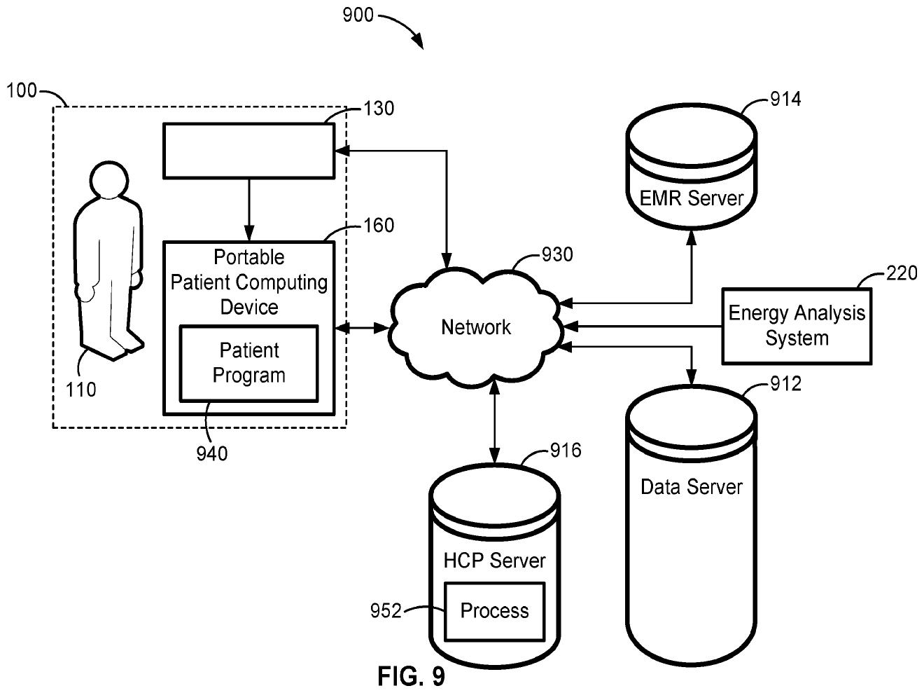

FIG. 9 is a block diagram of an example health care system 900 for obtaining data from patients using the energy monitoring in the home 100 shown in FIG. 1. The health care system 900 includes a data server 912, an electronic medical records (EMR) server 914, a health or home care provider (HCP) server 916, patient computing device 160, and the analysis system 200 in FIG. 2. The patient computing device 160 is co-located with the patient 110 and the energy monitor 130 is installed in the home 100 in this example. In the system 900, these entities are all connected to, and configured to communicate with each other over, a wide area network 930, such as the Internet. The connections to the wide area network 930 may be wired or wireless. The EMR server 914, the HCP server 916, and the data server 912 may all be implemented on distinct computing devices at separate locations, or any sub-combination of two or more of those entities may be co-implemented on the same computing device.

The patient computing device 160 is configured to intermediate between the patient 110 and the remotely located entities of the system 900 over the wide area network 930. As explained above, the energy monitor 130 may subsume some or all of the functions of the patient computing device 160 and directly communicate with any of the servers in the system 900. In the example implementation of FIG. 9, this intermediation is accomplished by a software application program 940 that runs on the patient computing device 160. The patient program 940 may be a dedicated application referred to as a “patient app” or a web browser that interacts with a website provided by the health or home care provider. The system 900 may include other energy monitors (not shown) associated with the hoes of respective patients who also have respective associated computing devices and associated HCP servers (possibly shared with other patients). All the patients in the system 900 may be managed by the data server 912.

As explained above, the data from the energy monitor 130 may be correlated with the health of the patient via the system 200. The health data from the system 200 may be supplied to the other servers of the system 700 such as the data server 912. The analysis module 220 in FIG. 2 may provide analysis of patient health based on any of the example techniques described above. The resulting health analysis from the analysis module 220 may be accessed by databases 950 accessed by any of the servers 912, 914, and 916.

In this example, the energy monitor device 130 may be configured to transmit the energy data from the home 100 to the patient computing device 160 via a wireless protocol, which receives the data as part of the patient program 940. The patient computing device 160 then transmits the energy data to the data server 912 and/or the system 200 according to pull or push model. The data server 912 or the system 200 may receive the physiological data from the computing device 160 according to a “pull” model whereby the computing device 160 transmits the energy data in response to a query from the data server 912 or the system 200. Alternatively, the data server 912 may receive the energy data according to a “push” model whereby the computing device 160 transmits the physiological data to the data server 912 or the system 200 on a periodic basis. As explained above, the system 200 may make such energy data available to the data server 912 for analysis in relation to producing health condition data of the patient. Further, the data server 912 may access databases such as the database 950 to store collected and analyzed data.

Data received from the patient computing device 160 is stored and indexed by the data server 912 so as to be uniquely associated with the patient 110 in the system 900. In this regard, although only one home 100 is illustrated in FIG. 9 for ease of explanation, the system 900 may include multiple energy monitor modules in different homes associated with different patients. The data server 912 may be configured to calculate summary data for each patient or home. The data server 912 may also be configured to receive data from the patient computing device 160 including data entered by the patient 110, behavioral data about the patient, or other relevant data.

The EMR server 914 contains electronic medical records (EMRs), both specific to the patient 110 and generic to a larger population of patients with similar disorders to the patient 110. An EMR, sometimes referred to as an electronic health record (EHR), typically contains a medical history of a patient including previous conditions, treatments, co-morbidities, and current status. The EMR server 914 may be located, for example, at a hospital where the patient 110 has previously received treatment. The EMR server 914 is configured to transmit EMR data to the data server 912, possibly in response to a query received from the data server 912.

In this example, the HCP server 916 is associated with the health/home care provider (which may be an individual health care professional or an organization) that is responsible for the patient's respiratory therapy. An HCP may also be referred to as a DME or HME (domestic/home medical equipment provider). The HCP server 916 may host a process 952 to transmit data relating to the patient 110 to the data server 912, possibly in response to a query received from the data server 912.

In some implementations, the data server 912 is configured to communicate with the HCP server 916 to trigger notifications or action recommendations to an agent of the HCP such as a nurse, or to support reporting of various kinds. Details of actions carried out are stored by the data server 912 as part of the engagement data.

The HCP server process 952 may include the ability to monitor the patient 110 in accordance with compliance rules that specify the required treatments or activities over a compliance period, such as 30 days. The summary data post-processing may determine whether the most recent time period is a compliant session by determining adherence to the compliance rule. The results of such post-processing are referred to as “compliance data.” Such compliance data may be used by a health care provider to tailor therapy that may include the inhaler and other mechanisms. Other actors such as payors may use the compliance data to determine whether reimbursement may be made to a patient. The HCP server process 952 may have other health care functions such as determining overall use or effectiveness of treatment based on collection of data from numerous patients. The process 952 may be able to determine the effectiveness of the medical devices through the collected data. For example, if patients are compliant but not improving, it may not be just an ineffective treatment for that patient but also an ineffective product overall. Additionally, if specific products have low compliance, it could be because of a fault with the product or an issue with its use (ergonomics or user interface, for example) that could encourage the health care entities to choose a different medical device. As explained above, the data may be correlated with geographically linked data to determine outbreaks in specific locations or increased probability of problems with patients based on nearby patients. For example, high temperatures or high pollen count could trigger specific health conditions of many similar patients in a specific area.

As may be appreciated data in the data server 912, EMR server 914, and HCP server 916 is generally confidential data in relation to the patient 110. Typically, the patient 110 must provide permission to send the confidential data to another party. Such permissions may be required to transfer data between the servers 912, 914 and 916 if such servers are operated by different entities.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A system for monitoring the health of a patient in a residential environment, the system comprising:
an energy monitoring module in communication with an electrical panel providing power to a plurality of electrical devices in the residential environment, the energy monitoring module operable to monitor energy consumed by the plurality of electrical devices;
a data energy analysis engine that collects energy data from the energy monitoring module over a period of time; and
a health data correlation engine that determines a health condition of the patient based on the collected energy data.

2. The system of claim 1, wherein the health condition includes one of a change in sleep quality or duration of the patient a change in diet or appetite of the patient a change in physical activity or mobility of the patient or a respiratory condition.

3. (canceled)

4. (canceled)

5. The system of claim 1, wherein the plurality of devices includes a therapeutic device for treatment of the patient.

6. (canceled)

7. The system of claim 1, wherein the energy data includes a pattern based on electrical usage.

8. The system of claim 1, wherein the energy data includes the times when each of the plurality of devices is turned on.

9. The system of claim 8, wherein the energy data includes determination of a specific device signature for each of the plurality of devices.

10. The system of claim 9, wherein the specific device signature is determined from machine learning.

11. The system of claim 9, wherein the specific device signature is determined from a rule set of device signatures.

12. The system of claim 9, wherein the correlation between the health condition and the specific device signature is determined from machine learning.

13. The system of claim 1, wherein the energy data includes the total electrical usage of the plurality of devices over a period of time.

14. A method for monitoring health of a patient in a residential environment, the method comprising:
collecting energy use data of at least one device in the residential environment;
determining an energy use pattern from the energy use data; and
correlating the energy use pattern with a health condition of the patient.

15. The method of claim 14, wherein the health condition includes one of a change in sleep quality or duration of the patient a change in diet or appetite of the patient a change in physical activity or mobility of the patient or a respiratory condition.

16. (canceled)

17. (canceled)

18. The method of claim 14, wherein the device is a therapeutic device for treatment of the patient.

19. (canceled)

20. The method of claim 14, wherein the energy use pattern is a pattern based on electrical usage.

21. The method of claim 14, wherein the energy use pattern includes the times when each of the plurality of devices is turned on.

22. The method of claim 21, wherein the energy data includes determination of a specific device signature for each of the plurality of devices.

23. The method of claim 22, further comprising determining the specific device signature from machine learning.

24. The method of claim 22, further comprising determining the specific device signature from a rule set of device signatures.

25. The method of claim 22, further comprising determining the correlation between the health condition and specific device signature from machine learning.

26. The method of claim 14, wherein the energy use pattern includes the total electrical usage of the device over a period of time.
